(19) United States
(12) Patent Application Publication
(10) Pub. No.: US 2014/0046234 A1
(43) Pub. Date: Feb. 13, 2014

(54) Dynamic Load Bearing Shock Absorbing Exoskeletal Knee Brace

(71) Applicant: Egas Jose-Joaquim DeSousa, Grand Blanc, MI (US)

(72) Inventor: Egas Jose-Joaquim DeSousa, Grand Blanc, MI (US)

(21) Appl. No.: 13/555,165

(22) Filed: Aug. 9, 2012

(51) Int. Cl. A61F 5/01

(52) U.S. Cl. 602/16; 602/26

(57) ABSTRACT

The exoskeletal dynamic load bearing shock absorbing knee brace makes use of the energy absorbing characteristics of specifically designed industrial shock absorbers which are held precisely in place by an articulated dynamic exoskeletal structure that is to be secured to the lower limbs of the individual with the injured knee. The exoskeletal structure is designed using the principles of the overcenter linkage to translate and transfer to the shock absorbers a representative fraction of the normal and extra normal ambulatory movements of the lower limbs of the user which makes possible for these shock absorbers to absorb a corresponding amount of energy and provide an alternate load bearing structure parallel to the knees thus introducing a desirable degree of protection for the injured knee.

This is continuation in part of the provisional application # US61/572,890 with filing date Jul. 25, 2011.

### FIELD OF THE INVENTION

This invention is in the field of orthotics. Specifically it provides for a knee orthotic device incorporating external shock absorbers appropriately designed to supplement the shock absorbing function of the impaired meniscus in the knee joint.

### BACKGROUND OF THE INVENTION

Injuries to the human knee joint are all too common resulting from participation in extreme sporting activities or resulting from accumulated damage with advancing years.

The knee joint is the biggest joint in the human body and is subject to various failure modes. One of the failure modes that this present invention addresses is the failure of the meniscus to maintain its integrity under sudden load spikes or accumulated damage. The meniscus is an avascular cartilage that acts as a shock absorber inside the knee. There are two disc shaped menisci in each knee.

FIG. 1 shows a representation of the knee with the meniscus between the Femur and the Tibia.

The damage resulting from meniscus tears and other failure modes is hard to contain and control under normal everyday working load situations. As the meniscus failure progresses the Femur and the Tibia come in direct contact resulting in irreversible damage under very painful conditions. Eventually, the afflicted individual in most cases, unable to bear the pain, will ask for and receive artificial knees depending upon the circumstances.

The present invention provides a load bearing shock absorbing device that could have a role in preventing further damage and help in the healing of the damaged knee joint. It could also be used to enable, otherwise impaired individuals to continue on with their active prior lifestyle

A Sep. 11, 2008 study in the New England Journal of Medicine titled Incidental Meniscal Findings on Knee MRI in Middle-Aged and Elderly Persons by seven physicians in the New England area concluded that “Incidental Meniscal findings on MRI of the knee are common in the general population and increase with increasing age.” The study reports Meniscal tear prevalence rates of 15% to 30% in women and men aged 50-59 and 27% to 37% rates in women and men aged 60-69. The incidence rates increase to a maximum of 50% with age advancing to 70-90 years.

A lot of these people learn to live with the pain. It is one of the objectives of this invention to help ameliorate the painful conditions.

### BRIEF OVERVIEW OF THE PRIOR ART

Most knee braces and supporting orthotics currently available in the marketplace are not targeted at providing the load bearing, shock absorbing function that is the key feature of the present invention.

A comprehensive search of available knee braces uncovers many very well built devices meant to support the injured knees. But these knee braces do not provide the load bearing and shock absorbing function of the present device.

A Patent search revealed the following prior art that is somewhat relevant to the present invention:
1. U.S. Pat. No. 4,688,599 Vito et al. This one is designed to provide stability in cases of loss of neuro-muscular control of a knee joint or a hip joint.
2. U.S. Pat. No. 5,645,524 Doyle. A knee support for supporting an injured knee while permitting bending and straightening of such a knee.
3. U.S. Pat. No. 5,352,190 Fischer et al. This provides for an apparatus to be used in bracing or exercising the knee joint in a manner that allows the bending of the knee joint only along a predetermined path which approximates the bending of the joint.

None of the above inventions provide the capability that the present invention provides: The protection of a load bearing and shock absorbing exoskeletal device worn around the lower limb specifically to reduce the loads and impacts on the knee joint.

### SUMMARY OF THE PRESENT INVENTION

The invention provides for a load bearing shock absorbing device that is to be attached to the lower limbs so that it may reduce the load and the shock loads experienced by the knee joint as the individual uses the legs to walk or run. Essentially it comprises a pair of upper struts designed to be attached to the upper limb or thigh by means of a sleeve made of strong and flexible material incorporating specially designed straps provided with Velcro or other buckle fasteners. This sleeve takes advantage of the somewhat conical shape of the human thigh to enable the desired load transfer from the sleeve to the thigh without the use of uncomfortably tight fits. The ends of the above named struts are pivotally attached via a clevis pin mechanism to a pair of shock absorbers. The shock absorbers are specified for this application based on required energy absorption capabilities with respect to the effective impact weight of the individual using the device, shock absorber stroke, internal return spring force and ease of integration in the present invention design. The shock absorbers are retained in a load bearing yet adjustable manner in a lower strut section that is to be strapped to the lower limbs or the calf and inserted via a quick disconnect mechanism into a suitably designed hinged mechanism that is made part of a specially designed and built shoe. All the straps used for this device are to be made of a strong yet flexible and durable fabric and provided with Velcro or any other buckle fasteners at the ends.

The overcenter linkage principle that is being used to actuate the shock absorbers utilizing the natural movement of the limbs can be best be explained by reference to the diagrams in FIGS. 2 and 3. In these diagrams 701 and 704 represent two points at which this device is strapped to the limb: The point in the thigh where the strap is attached and the point in the shoe where the lower strut is attached; The center point of the clevis pin in the shock absorber is represented by 702 while 703 represents the natural center of the knee. With the individual in the seated position the length of the linkage 701-702-704 is greater that the length of the path in the leg 701-703-704.

Thus, when the individual stands up in the vertical position for instance, the movement of the leg forces the linkage 701-702-704 to decrease in length thereby forcing the shock absorber to close in until the new linkage length equals the length of the path in the leg 701-703-704. In this position the shock absorber is primed for action and the return springs push up against upper sleeve as far as the fastening conditions permit. As the individual walks or runs the movement of the limbs introduces sufficient movement to the shock absorbers to elicit a force response which helps in reducing the load on the knee. The shock absorber selected has a force response directly proportional to the rate of change of position of its piston thus more vigorous and faster movements elicit a greater force response, and this helps reduce the load on the knee under shock and faster movement conditions.

Three design options are presented in this application for Patent. The load bearing and shock absorbing feature remains constant for all three options. What changes is the design of the lower limb struts and the design of the incorporation of the connector to the shoe or boot. In the preferred design option the lower strut has a single hinged insertable connection to the back of the heel. This design allows the greatest degree of freedom for the foot. The other two options required two lateral hinged connections to the shoe component. One design uses two parallel lateral struts which provide some degree of freedom to the foot, while as the last design using a single assembly connecting the shock absorbers to the shoe mechanism provides a good degree of support and has some aesthetic advantages.

Many design changes and improvements will become obvious to those schooled in the arts based on these disclosures. The present descriptions are to be viewed as more illustrative of the embodied principles rather than specific design guidelines.

### HOW THIS IS TO BE USED

The individual puts on the specially designed shoe with the load bearing insert in the seated position. In this position the individual inserts the lower strut assembly bushings on to the shoe support hinged pin. He can at this point strap the lower linkage to his calf with the strap provided by fastening snugly the straps fitted with the buckle or the Velcro brand fasteners.

At this point he rolls the cuff detail snugly over his tight and fastens it tight with the two straps provided using the buckle or the Velcro fasteners. The axis of the clevis pin of the shock absorber should be a certain distance away from the natural axis of the knee based on the design of the exoskeletal brace.

As the individual stands up he can feel the upper cuff snug up tight as the shock absorber is actuated and the return spring pushes the clevis end up.

By walking back and forth the individual can feel the shock absorber pick up a good part the load on the knee. This results in immediate relief from the pain as the femur and tibia contact is reduced.

### BRIEF DESCRIPTION OF THE DRAWINGS

With reference to the attached drawings in which like reference numbers refer to like parts:

FIG. 1 show a representation of the human knee joint with the Omeniscus in between the Femur and the Tibia.

FIG. 2 and FIG. 3 show a representation of the “overcenter” linkage principle that is being used in this invention.

FIG. 4 shows an isometric view of the preferred design with the strut support at the back of the leg. For the sake of clarity the device is shown in isolation and only the sole of the shoe is depicted for directional input. This view shows the device as it would be with the individual getting ready to use it in a seated position.

FIG. 5 shows the same device of FIG. 4 but as it would be if the individual were to stand up with the device on.

FIG. 6 shows an isometric view of an optional design with lower limb lateral supports

FIG. 7 shows an isometric view of another optional design with the lower supports in front of the tibia.

FIG. 8 shows an isometric view of the strap used to snug up the lower support assembly of the preferred rear support knee brace to the calf of the individual.

FIG. 9 shows an isometric view of the lower support subassembly of the preferred rear support knee brace.

FIG. 10 shows an isometric view of the lower subassembly of the frontal support option of the knee brace showing how the strut assembly is connected to the shoe insert made integral part of the shoe sole.

FIG. 11 shows an isometric view of the lower subassembly of the lateral support option of the knee brace also showing how the lateral struts are connected to the shoe insert that has been made an integral part of the shoe sole.

FIG. 12 shows an isometric view of the sleeve assembly.

FIG. 13 shows a cross sectional view of the sleeve illustrating how the Velcro fasteners are used in the straps to secure the sleeve snugly to the upper limb of the individual.

FIG. 14 shows an isometric view of the shock absorber assembly.

FIG. 15 shows an isometric view of the upper strut or bar with the clevis end and the holes used to fasten it to the sleeve.

FIG. 16 shows an isometric view of the shoe sole being used in the first design option with the load bearing insert in place in the heel area.

FIG. 17 is an isometric view of the pivoted pin that is attached to the shoe insert and that connects to the lower assembly of the preferred rear support knee brace.

FIG. 18 shows an isometric view of the shoe insert part also used in the preferred first design option.

FIG. 19 shows an isometric view of one of the straps used to snug up the sole assembly to the ankle of the individual

FIG. 20 shows an isometric view of the shoe insert used in the design options with the lateral support and the frontal support. This Fig also shows in place the two quick disconnect details 24 which will be attached via the pivot pins at 241 to the lower strut assemblies.

FIG. 21 shows an isometric view of the optional comfort pad that is to be used along with the upper struts.

### DETAILED DESCRIPTION OF THE INVENTION

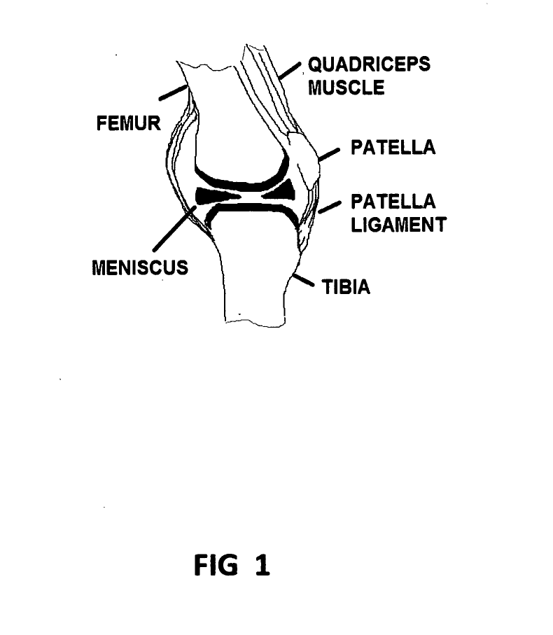

FIG. 1 shows a diagram of the lateral view of the human knee. The meniscus cartilage is represented in the cross sectional view as triangular bodies that help keep the Femur and Tibia in place and act as internal shock absorbers. The failure mode of the meniscus generally manifests in tears to the meniscus body which gradually deteriorates and the resulting contact between the Femur and the Tibia leads to sequence of events that cause a lot of pain and tend to force the afflicted individual to refrain from many of their normal ambulatory activities.

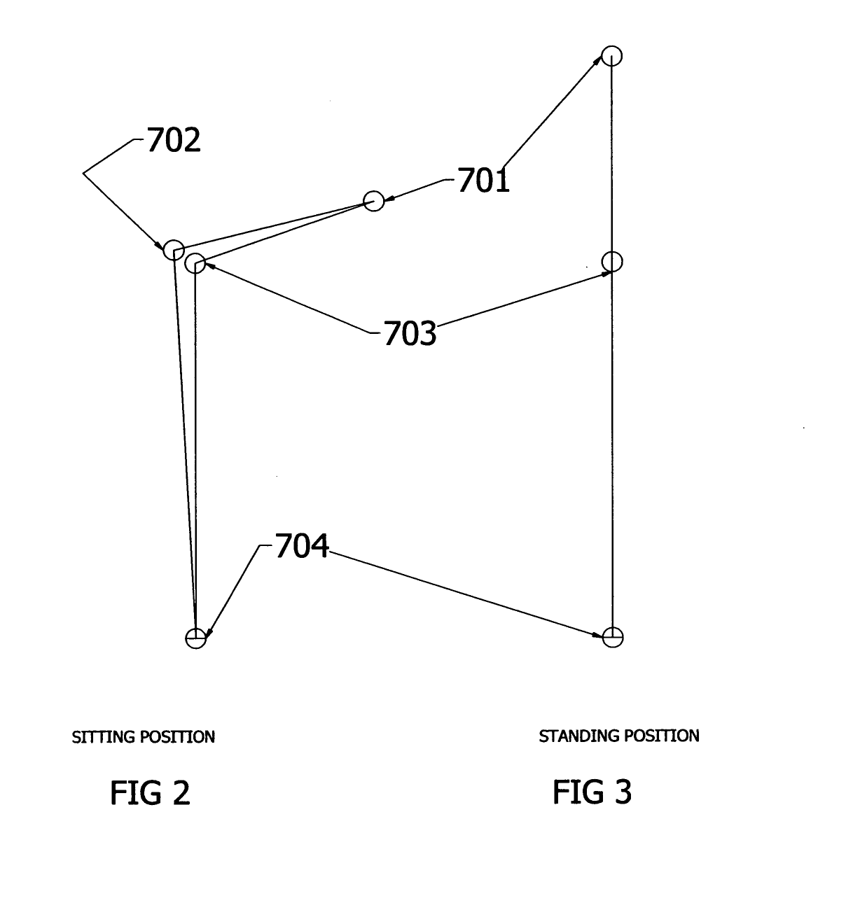

FIGS. 2 and 3 show a diagram used to describe the overcenter linkage principle that is being used in the present invention, and can also be observed in many everyday applications like clamps and buckles.

The overcenter principle is being described with reference to FIGS. 2 and 3 in the Summary section and is inserted here again for the sake of convenience: The overcenter linkage principle that is being used to actuate the shock absorbers utilizing the natural movement of the limbs can be best be explained by reference to the diagrams in FIGS. 2 and 3. In these diagrams 701 and 704 represent two points at which this device is strapped to the limb: The point in the thigh where the strap is attached and the point in the shoe where the lower strut is attached; The center point of the clevis pin in the shock absorber is represented by 702 while 703 represents the natural center of the knee. With the individual in the seated position the length of the linkage 701-702-704 is greater that the length of the path in the leg 701-703-704.

Thus, when the individual stands up in the vertical position for instance , the movement of the leg forces the linkage 701-702-704 to decrease in length thereby forcing the shock absorber to close in until the new linkage length equals the length of the path in the leg 701-703-704. In this position the shock absorber is primed for action and the return springs push up against upper sleeve as far as the fastening conditions permit. As the individual walks or runs the movement of the limbs introduces sufficient movement to the shock absorbers to elicit a force response which helps in reducing the load on the knee. The shock absorber selected has a force response directly proportional to the rate of change of position of its piston thus more vigorous and faster movements elicit a greater force response, and this helps reduce the load on the knee under shock conditions and faster movement conditions.

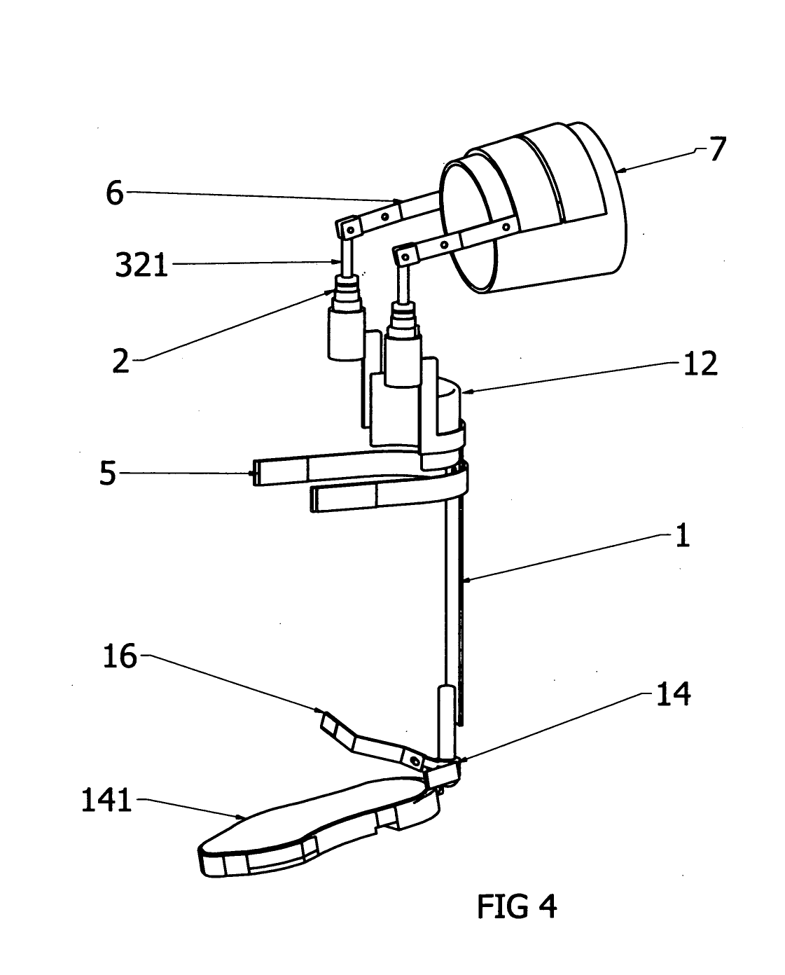

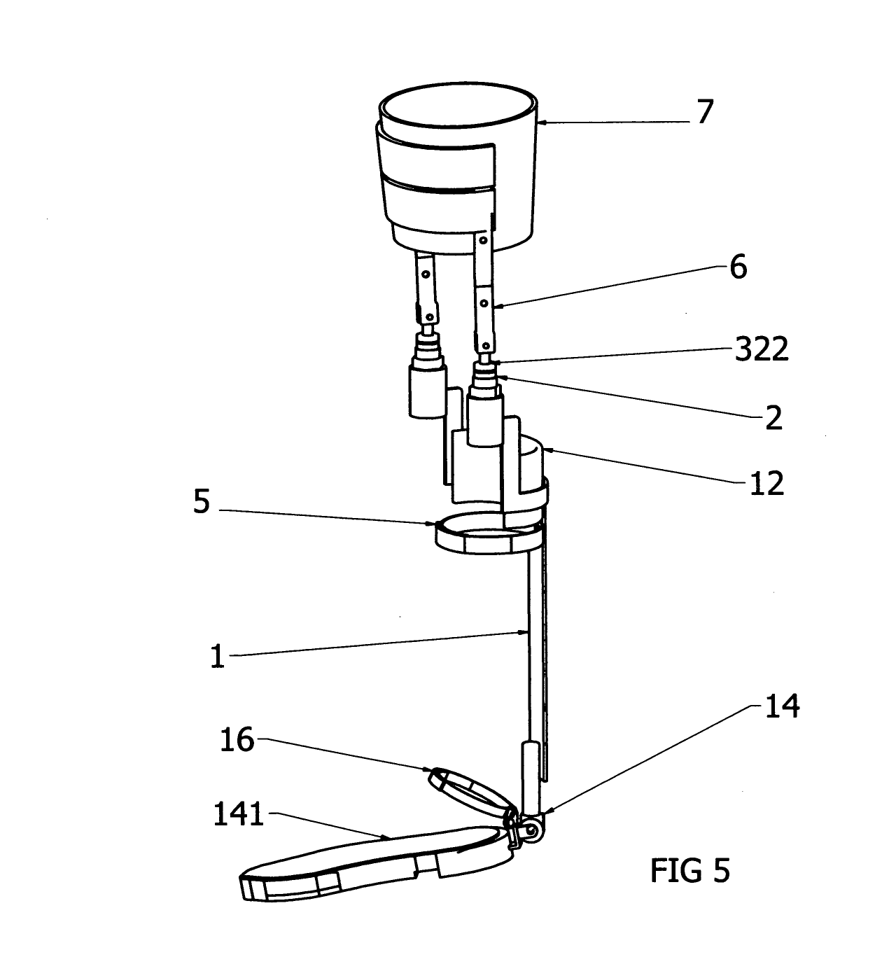

FIG. 4 shows an isometric view of the isolated preferred exoskeletal load bearing, shock absorbing knee brace with the individual in the seated position. The person who is to wear the brace is not shown in the drawing but the position of the shoe sole 141 helps infer the actual position of the leg as it would be with the thigh wrapped with the sleeve 7 and to be fastened with the strap 5 at the calf and straps 16 at the ankle. Note the shock absorber 2 with the fully extended rod 321. FIG. 5 shows an isometric view of the device in FIG. 4 but with the individual in standing position with the straps 5 and 16 wrapped around the calf and the ankle and the shock absorber in the pushed in position as depicted by the shock absorber rod rod positions 322.

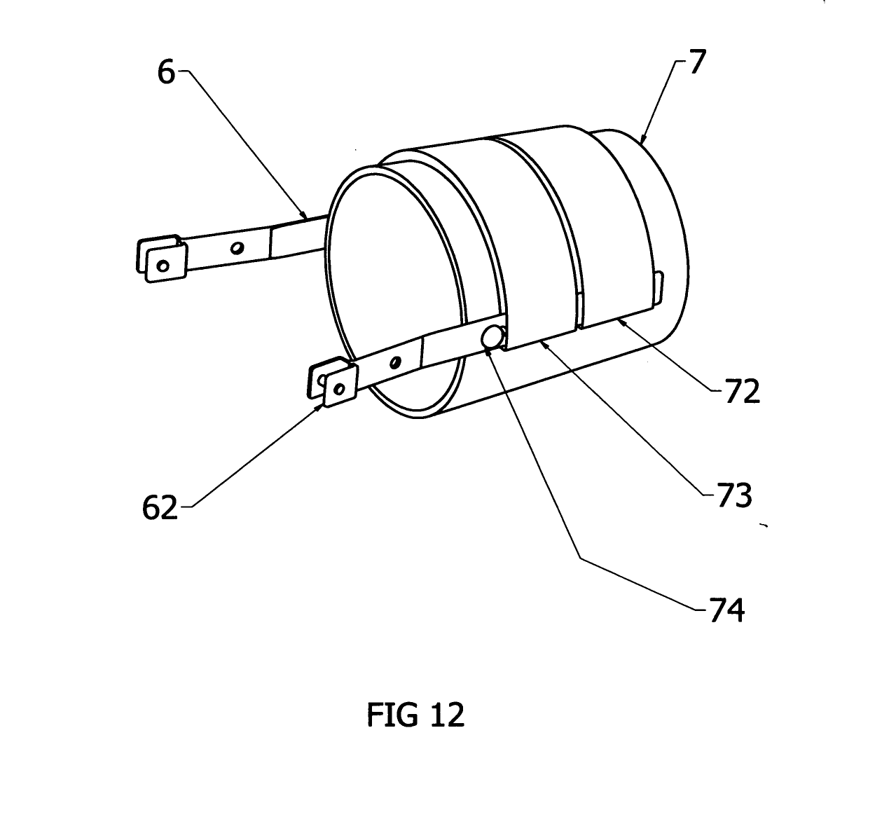

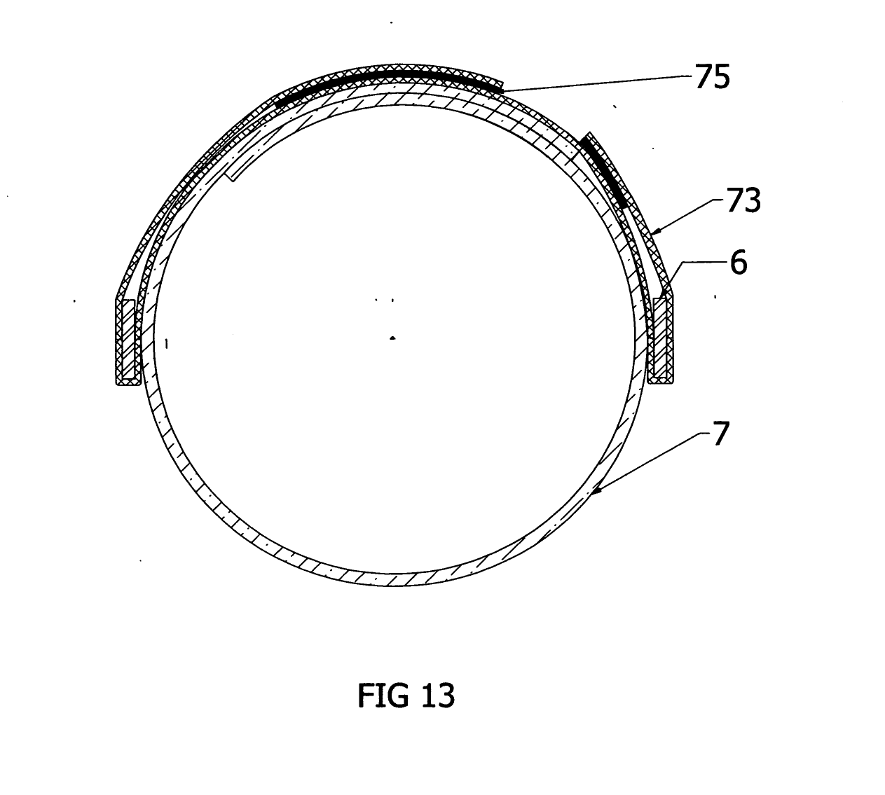

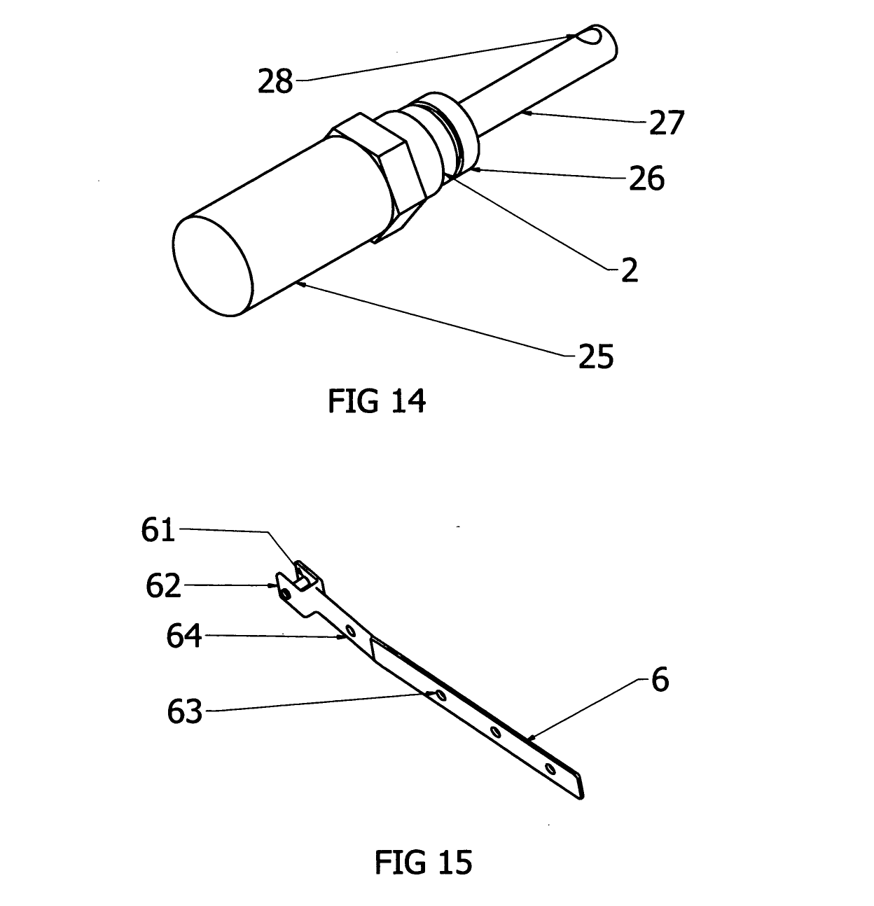

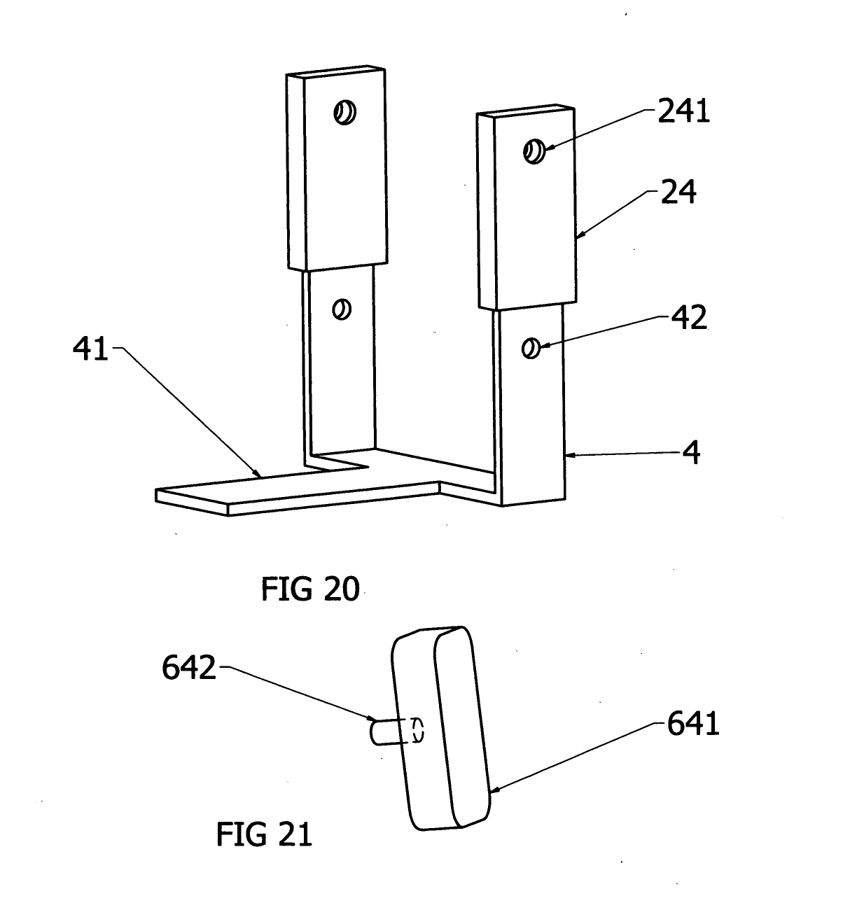

The upper thigh sleeve assembly 7 is also shown as an isometric view in FIG. 12, and in a cross sectional view in FIG. 13 demonstrating the design using a Velcro brand hook and loop fastener 75. With reference to these drawings 6 represents the upper struts also shown in isolation in FIG. 15 with the holes 63 used to attach it using fasteners 74 to the sleeve 7. The sleeve 7 is made out of a flat material that is strong, flexible and light. The struts 6 are fastened to the sleeve 7 is such a manner that when the sleeve is wrapped around the thigh of the individual the struts 6 present the clevis ends 62 for insertion of the pins 61 to enable a pivotal connection to the rod end 28 of the shock absorbers 2 also shown in isolation in FIG. 14. The strut in FIG. 15 also is provided with appropriately located holes 64 for the attachment of optional comfort pads 641 shown in isolation in FIG. 20 by use of the fastener 642. Coming back to the description of the sleeve assembly 7 the cross sectional view shows the straps 73 wrapped around the struts 6 which are fastened to the sleeve 7 using spacers and fasteners 74, in such a way that the sleeve can be held snugly in place by use of the Velcro fasteners represented by 75 also in FIG. 13.

The sleeve 7 is to be made of a strong yet flexible material such a 4 ply food conveyor belting material lined suitably with a soft yet somewhat sticky layer for comfort of the user. The sleeve 7 is to be wrapped around the thigh or upper limb in a shape that is conical as shown in FIGS. 4, 5, 6 and 8 to fit the shape of the average human thigh. In FIG. 21, 641 represents the soft yet durable part of the comfort pad that is to be adjusted to push comfortably against the upper leg of the individual.

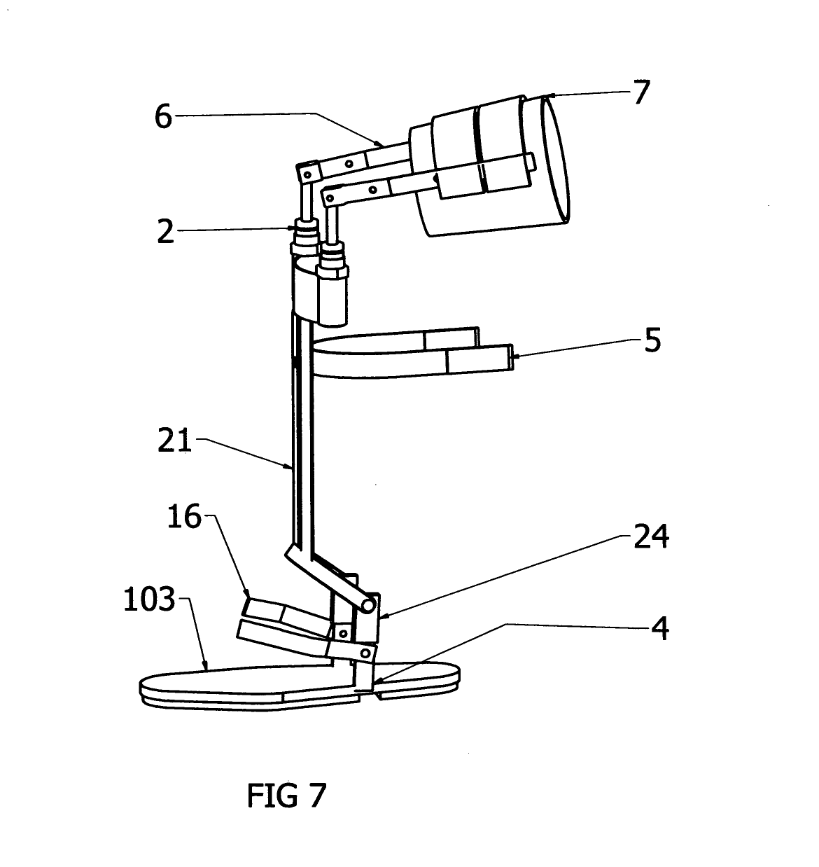

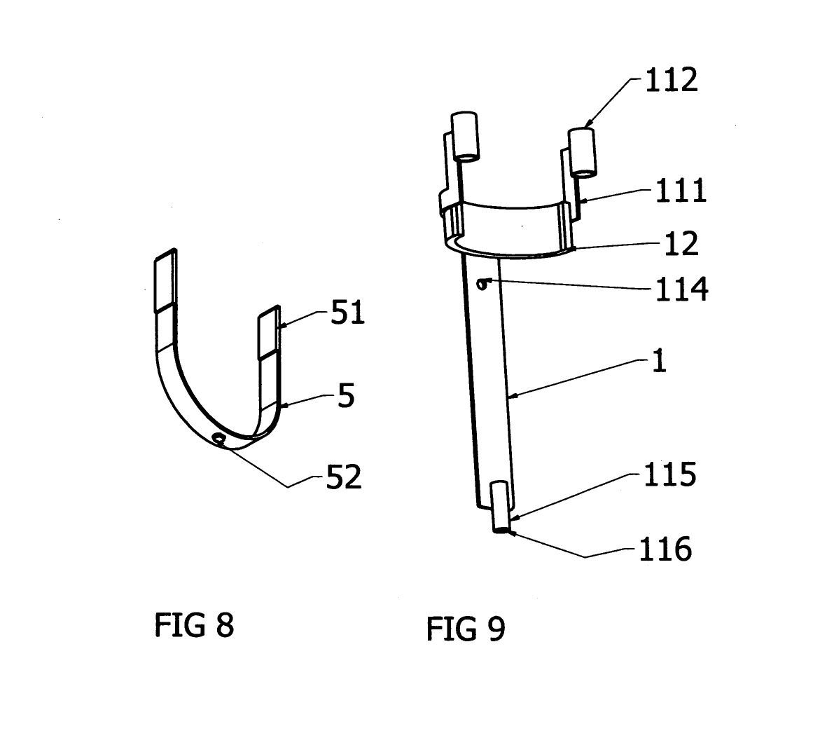

FIG. 4 also shows the shock absorber 2 provided externally with a vertical position mounting adjustment mechanism in the form of a threaded body and a nut 25 as shown in the isolated view in FIG. 14. The shock absorbed is retained in the sleeve 112 shown in FIG. 9 made part of the lower strut subassembly 1 and also shown in isolation in FIG. 7. FIG. 7 also shows the sleeves 112 being made part of a strut 111 that starts out as a semicircular detail made part of the lower strut detail 1 by welding or other means of fixed attachment. The lower strut detail 1 shown in FIG. 9 is made of a strong yet light material and carries at the lower end a sleeve or bushing 115 with an internal hole 116 suitably dimensioned to accept the moveable pin 171 a part of detail 17 which is shown in FIG. 17 in isolation. This pin is attached to the lower shoe insert 14 using the clevis 142 which is an integral part of the load bearing shoe insert 14 shown in isolation in FIG. 18. The pin 17 to the shoe insert 14 pivotal connection is designed such that the pin 171 has a limited amount of movement around the axis of the clevis pin hole 173. FIG. 17 also shows the load bearing shoulder 174 of the pin detail 17. This is what supports the sleeve detail 115 of the lower strut assembly 1 described above. FIG. 9 also shows the hole 114 on the strut 1 used to attach the strap 5 shown in FIG. 4 and as an isolated isometric view in FIG. 8 This is the strap that is provided with Velcro band fasteners represented by 51 and the attachment hole 52 used to snug up the brace to the calf of the user.

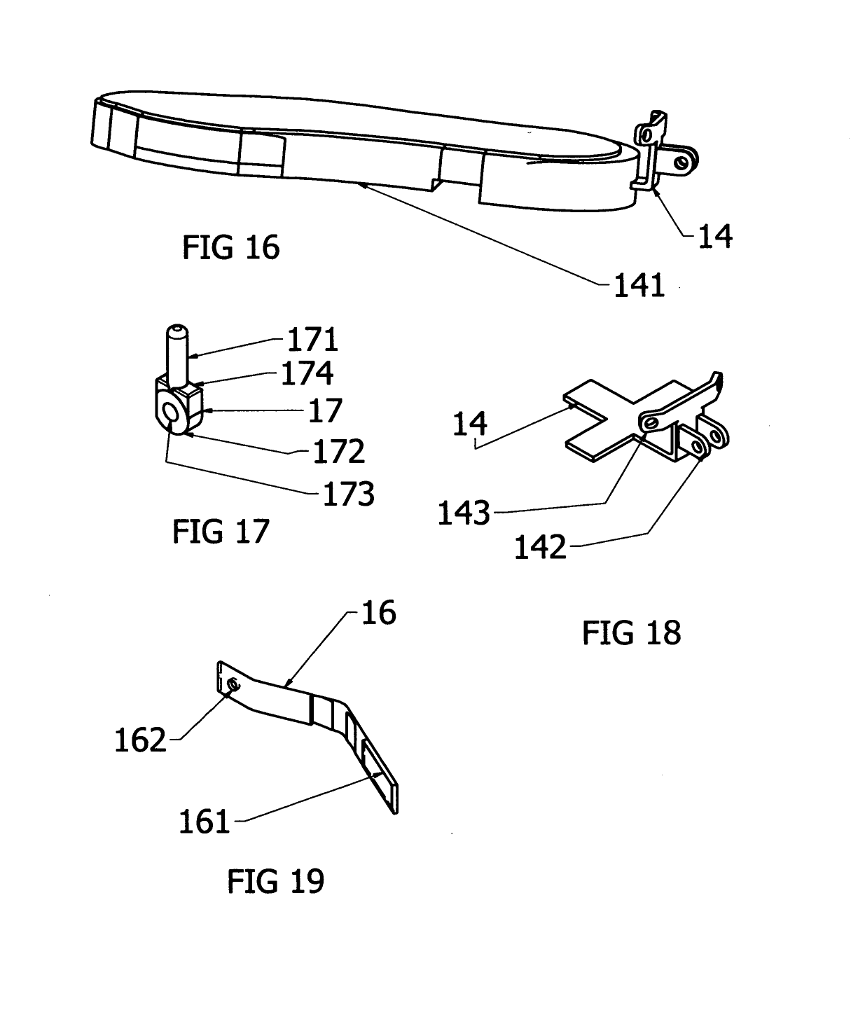

FIG. 4 also shows the sole of the shoe 141 thus depicted for the sake of clarity and simplicity. The sole 141 represents the position of the foot of the individual and a detailed isometric view of the same sole is shown in FIG. 16. The sole may be made of a customary shoe sole material such as rubber or leather or a synthetic composite material. FIG. 16 shows how the shoe insert made of a strong load bearing material such as a light metal or high density plastic, is to be incorporated in the heel of the sole of the shoe. It is to be inserted in the middle of the heel and fixed in place so that it becomes an integral part of the sole.

FIGS. 4 and 5 also show the straps 16 used to support the shoe insert around the ankles. The strap 16 is also shown in isolation in FIG. 19 with the Velcro fastener represented by 161 and attachment hole 162 used to fasten it strongly to the shoe insert 14 at the holes 143 as shown in the isolated isometric view in FIG. 18.

FIG. 4 also shows the soft contact liner 12 attached to the lower strut 1 fixedly layered on detail 111 as depicted in FIG. 9.

The design is to be customized to the dimensional requirements of individual users so as to maximize the shock absorber impact.

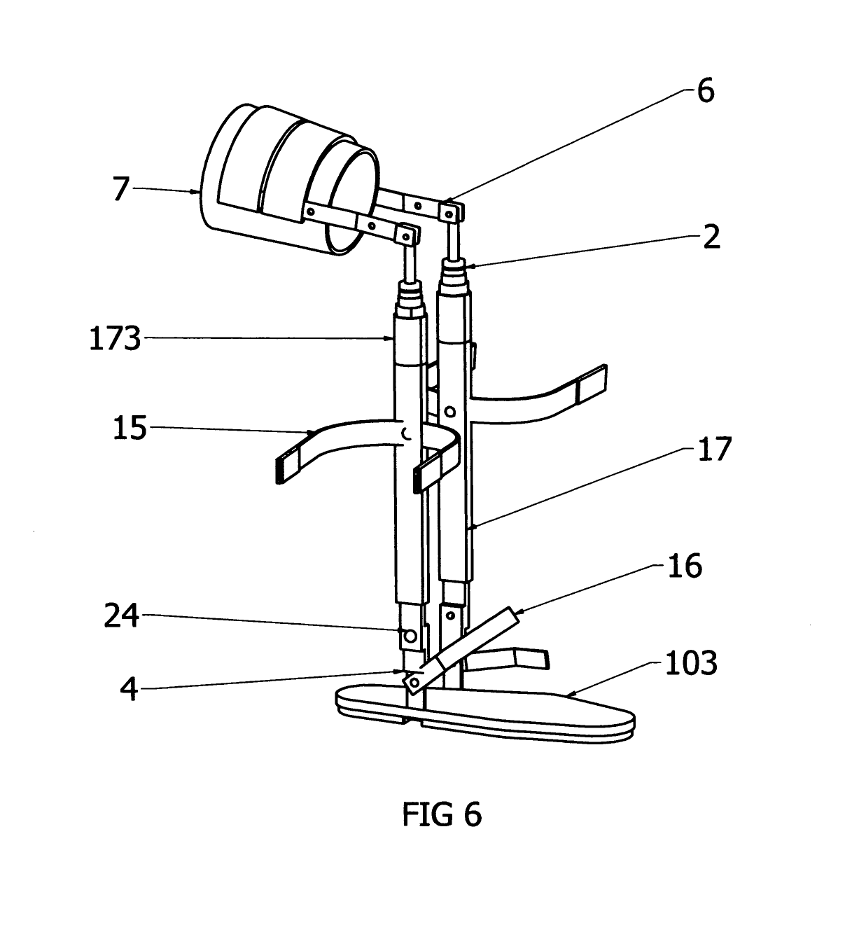

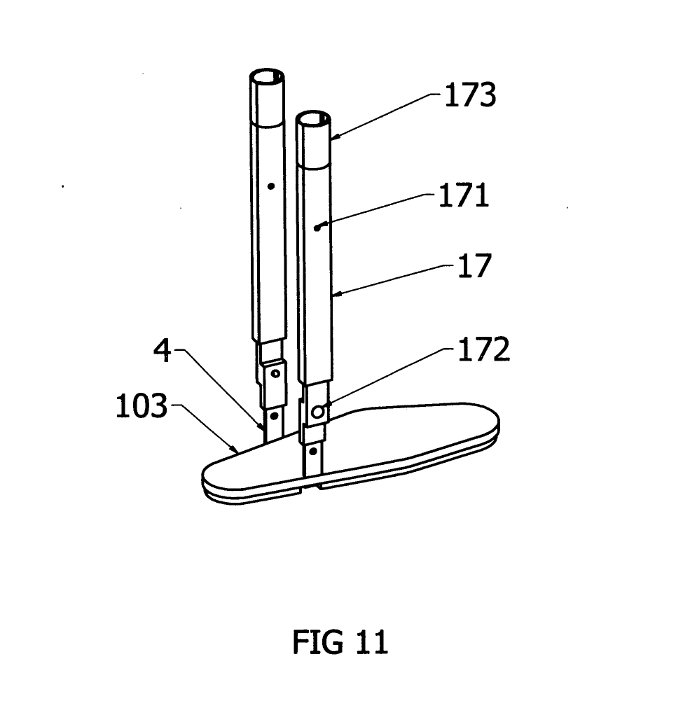

FIG. 6 shows an isolated isometric view of second knee brace design option with the lower struts positioned to the sides of the lower leg. The sleeve assembly 7 is pivotally attached via the upper struts 6 to the shock absorber 2 as described earlier under option 1. The pair of lateral struts 17 also shown in the FIG. 11 have at the top end, been provided with retaining sleeves 173 used to hold the shock absorbers 2 in place. The same threaded body and nut shock absorber positional adjustment mechanism described for the design option 1 is being used here. At the bottom end, the struts 17 are attached via a pivot pin 172 to the shoe insert 4 shown integrated with shoe sole 103 in FIG. 11. The shoe retainer 4 is also being shown in an isolated isometric view in FIG. 20. Here we see the base 41 of the insert 4 which is integrated in the heel of the sole. Also shown in this figure is the detail 24 which provides the quick disconnect feature of this option. This detail is designed to slide on and engage the uprights of the shoe insert 4. This detail 24 also is provided with a hole 241 designed to be used for the pivoting connection to the above described lower struts 17 with the pivot pin 172 shown in FIG. 11. Also shown in FIG. 11 are the holes 171 used for attachment of the straps 15 that are to be fastened using the Velcro fasteners provided to snug up the lower struts 17 around the calf of the user. FIG. 6 also shows the straps 16 attached to the shoe retainer 4 using holes 42 depicted in FIG. 20. The straps 16 are to be used to snug up the lower brace around the ankles of the user.

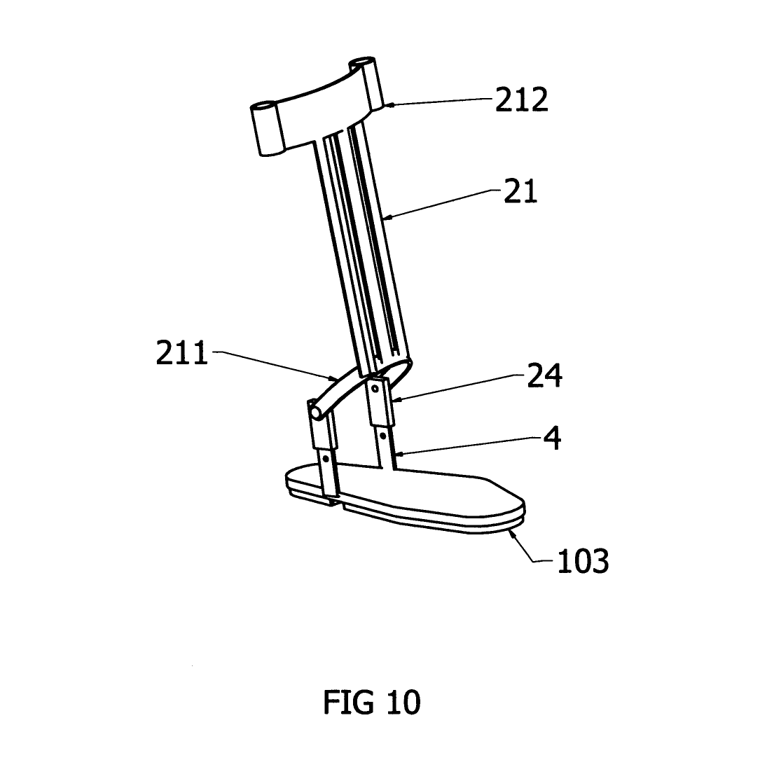

A third knee brace option design is being shown in FIG. 7. This option provides for a lower strut assembly 21 designed to be positioned in front of the lower leg. An isolated isometric view of the lower strut assembly is also being shown in FIG. 10. The shock absorber retainers 212 are positioned fixedly at the top of the vertical struts 21 which are fixedly attached to a an arched detail 211 designed to go around the ankle of the individual and connect to the verticals of the shoe insert 4 shown in FIG. 20 using pivot pins as also described earlier under the second design option. Shoe insert 4 is integrated in the shoe sole 103 as shown in FIG. 7 and FIG. 10. Also seen in FIG. 7 are the two straps 16 attached to the shoe insert 4 using holes 42, which are to be used to snug up the lower assembly to the ankles of the user. The lower strut assembly 21 is attached via pivot connection to the detail 24 which also provides the quick disconnect feature to the shoe insert 4 as described under the second option. The strap 5 described under option 1 and shown in isolation in FIG. 8 is attached to the lower strut assembly 21 and is used to snug up the lower assembly to the calf of the user. FIG. 7 also shows the straps 16. This third option while contributing to constraints on the lateral movement of the foot does provide solid support to the knee and adds a certain aesthetic flair.

1. An exoskeletal load bearing shock absorbing knee brace designed to support the knee principally by reducing the load on the knee and absorbing peak shock loads by use of a pair of specifically designed external shock absorbers held in working position by an upper sleeve assembly consisting of a pair of struts held parallel to the upper leg by the sleeve which is firmly cuffed to the upper part of the leg or the thigh and a lower strut assembly held in place parallel to the lower limb by cuffing to the calf and supported in a load bearing pivotal manner by a load bearing shoe sole insert which is made integral part of the shoe of the individual using said knee brace.

2. The exoskeletal load bearing and shock absorbing knee brace of claim one wherein the upper strut assembly and the lower strut assembly are pivotally connected to each other in such a way that with the individual using the brace in the seated position, the pivot point is at a certain distance away from the natural pivot point of the knee of the individual using the brace, and such that when the individual stands up, the distance differential between the two pivot points is to be overcome by the displacement of the shock absorber piston and the piston rod, and furthermore any relative angular movement between the upper and lower limbs is to cause a corresponding actuating displacement of the shock absorber piston thereby producing the associated shock absorber load response, said shock absorber being designed to respond to minute changes in displacement of the piston.

3. The exoskeletal load bearing and shock absorbing knee brace of claim one wherein the upper strut assembly and the lower strut assembly are dimensionally matched to the leg of the individual using said brace, so as provide the distance differential between the two pivot points required to activate the shock absorbers.

4. The exoskeletal load bearing and shock absorbing knee brace of claim one wherein the shock absorbers are designed to optimize the actuation requirements and the corresponding load response so that the load on the knees of the user is reduced as required.

5. The exoskeletal load bearing shock absorbing knee brace of claim two wherein the shock absorbers are provide with external return springs or preferably internal return springs that are capable of returning the shock absorber piston to the top of the stroke under no load conditions and also capable of pushing the connected upper sleeve snugly up against the upper thigh of the individual taking advantage of the conical shape of the lower part of the human thigh.

6. The knee brace of claim one wherein the upper struts are supported in a load bearing capable manner by the upper sleeve designed to fit the conical shaped human thigh, and held in place snuggly by straps thus enabling the transfer of load directly, bypassing the knee joint.

7. The exoskeletal load bearing shock absorbing knee brace of claim two wherein the lower strut assembly is located behind the lower limb and is provided at the top end with load bearing semicircular attachments that support the shock absorbers that are to be positioned to the front and sides of the knee joint and is provided at the opposite lower end with a fixedly attached bushing that can engage the pin supported via pivot pin by the shoe insert which is a detail that is made an integral part of the heel of the sole of the shoe.

8. The exoskeletal load bearing shock absorbing knee brace of claim seven wherein the loose fit between the bushing at the lower end of the lower strut assembly and the pin pivoted to the shoe insert is such that it allows a quick connect and disconnect as well as it allows the two details to move around freely around the cylindrical axis, yet is sufficient to maintain the connection in place at all times until disconnected by the individual using the brace.

9. The exoskeletal load bearing shock absorbing knee brace of claim seven wherein the lower strut assembly includes a quick disconnect feature for attachment to the lower limb in the calf area and a quick disconnect feature for attachment of the shoe insert assembly to the ankle of the individual.

10. The exoskeletal load bearing shock absorbing knee brace of claim seven wherein the load bearing shoe insert is made an integral part of the heel of the sole of the shoe such that it can transfer evenly to the ground the load being imposed on it by the lower strut assembly of the knee brace.

11. The exoskeletal load bearing shock absorbing knee brace of claim seven wherein the quick disconnect feature for attachment of the brace to the limbs may be a strap made out of flexible yet strong and durable material fitted with Velcro brand loop and hook fasteners or even a strap fitted with an overcenter buckle.

12. The exoskeletal load bearing shock absorbing knee brace of claim two wherein the lower strut assembly is designed to be located laterally with respect to the lower limb each of the two struts being provided with a means of retaining the shock absorber at the top end and at the lower end a pivotally connected quick disconnect sleeve to engage and retain the upright part of the shoe insert which is made an integral part of the sole of the shoe to be worn by the individual using said brace.

13. The exoskeletal load bearing shock absorbing knee brace of claim two wherein the lower strut assembly is designed to be located in front of the lower limb being provided at the top end with a means of retaining the shock absorbers in position to be pivotally connected to the upper limb struts and also being provided at the lower end with a means of retaining pivotally the quick disconnect sleeves that are to engage and retain the upright part of the shoe insert which is made an integral part of the sole of the shoe to be worn by the individual using said brace.
